(19) United States
(12) Plant Patent Application Publication
(10) Pub. No.: US 2016/0242338 P1
(43) Pub. Date: Aug. 18, 2016

(54) Grapevine named 'EARLY SAUVIGNON'

(50) ;

(71) Applicants: Universita degli Studi di Udine, Udine UD (IT); Istituto di Genomica Applicata, Udine UD (IT)

(72) Inventors: Simone Diego Castellarin, Udine (IT); Guido Cipriani, Faedis (IT); Gabriele Di Gaspero, Cividale del Friuli UD (IT); Michele Morgante, Tricesimo UD (IT); Enrico Peterlunger, Codroipo UD (IT); Raffaele Testolin, Udine (IT)

(73) Assignees: Universita degli Studi di Udine, Udine UD (IT); Istituto di Genomica Applicata, Udine UD (IT)

(21) Appl. No.: 14/545,098

(22) Filed: Mar. 26, 2015

(30) Foreign Application Priority Data
Feb. 13, 2015 (QZ) 2015/0368

(51) Int. Cl. A01H 5/00

(52) U.S. Cl. PLT/207

(57) ABSTRACT

A new and distinct variety of grapevine named ‘EARLY SAUVIGNON’, primarily adapted to the growing conditions of the temperate regions, and characterized by its strong vigor; erect growth habit; wedge-shaped leaves with medium texture; medium weight and conical shaped berry clusters; early maturing, firm berry flesh with no coloration and neutral taste primarily for winemaking; early harvesting time; and resistance to winter temperatures (to −22° C.), resistance to downy mildew and tolerance to powdery mildew.

### LATIN NAME OF THE GENUS AND SPECIES OF THE PLANT CLAIMED

Vitis×vinifera (hybrid)

### VARIETY NAME

‘EARLY SAUVIGNON’

### BACKGROUND OF THE INVENTION

The present invention relates to a new and distinct summer/fall bearing grapevine variety, botanically known as Vitis vinifera, and hereinafter referred to by the name ‘EARLY SAUVIGNON’.

The new grapevine ‘EARLY SAUVIGNON’ is a product of a controlled breeding program conducted by the inventors in Udine, Italy. The objective of the breeding program was to develop a new grapevine variety particularly characterized by resistance to cold (<−20° C.), resistance to downy mildew (Plasmopara viticola), and tolerance to powdery mildew (Uncinula necator).

The new grapevine ‘EARLY SAUVIGNON’ originated from a cross made by the inventors in 2002 in Udine, Italy. The female or seed parent is the grapevine variety Vitis vinifera ‘Sauvignon’ (unpatented), and the male or pollen parent is the grapevine variety Vitis ‘20/3’ (Bianca×SK77-4/5) (unpatented).

The new grapevine ‘EARLY SAUVIGNON’ was discovered and selected by the inventors as a single flowering plant within the progeny of the stated cross in a controlled environment in 2002 in Udine, Italy. Asexual reproduction of the new grapevine variety by grafting was first performed in February 2004 in Rauscedo, Friuli Venezia Giulia region, Italy, and has demonstrated that the combination of characteristics as herein disclosed for the new cultivar are firmly fixed and retained through successive generations of asexual reproduction. The new cultivar reproduces true to type.

### SUMMARY OF THE INVENTION

‘EARLY SAUVIGNON’ is primarily adapted to the climate and growing conditions of the temperate regions with average yearly temperature about 13° C., minimum winter temperature about −20° C., annual rainfall around 700-1500 mm of rain (e.g. North-Eastern Italy, Friuli). This region provides the necessary year-round temperatures required for it to produce and maintain a strong vigorous plant with consistent fruit production from April through November on primocanes and in the ensuing year from April through November on the floricanes.

The following traits have been repeatedly observed and are determined to be unique characteristics of ‘EARLY SAUVIGNON’, which in combination distinguish this grapevine plant as a new and distinct variety.
1. Strong vigor;
2. Erect growth habit;
3. Wedge-shaped leaves with a medium green upper surface and light green lower surface and a medium texture;
4. Fruit clusters with medium weight, conical shape, firm berry flesh with neutral taste, and no flesh coloration, early maturity (end of August in Middle Friuli, North-Eastern Italy); and primarily used for wine.
5. Primocanes and floricanes with an elliptic cross section, brownish color, no lenticels, and small internodes of 8 mm diameter;
6. Early harvesting time, at end of August in Middle Friuli, North-Eastern Italy; and
7. Resistance to winter temperature (−22° C.), resistance to downy mildew, and tolerant to powdery mildew, if overripening is allowed it is susceptible to cluster rots.

Plants of the new grapevine ‘EARLY SAUVIGNON’ differ from plants of the parents, Vitis vinifera ‘Sauvignon’ (unpatented) and Vitis ‘20/3’ (unpatented), in the following characteristics described in Table 1.

TABLE 1
Comparison with Parent Varieties

| | New Cultivar | Female Parent | Male Parent |
|---|---|---|---|
| Charac- | ‘EARLY | ‘Sauvignon’ | ‘20/3’ |
| teristic | SAUVIGNON’ | (unpatented) | (unpatented) |
| vigor | strong | medium | medium |
| growth | erect | semi-erect | semi-erect |
| habit | | | |
| leaf | medium, med- | medium, | Medium size, |
| | ium green col- | medium green | light green |
| | or (upper sur- | color (upper | color (upper |
| | face), light | surface), pale | surface), green |
| | green color | green color | color (lower |
| | (lower surface), | (lower surface), | surface), no |
| | no hairs in both | prostrate hairs | hairs in both |
| | surfaces, wedge- | on upper surface: | surfaces, wedge- |
| | shaped, medium | present, high den- | shaped-kidney- |
| | texture, | sity of prostrate | shaped, medium |
| | | hairs on lower | texture |
| | | surface, circular | |
| | | shape, medium | |
| | | texture | |
| cluster | medium weight, | very low weight, | low weight, con- |
| | conical with | cylindrical-coni- | ical, with 1-2 |
| | two wings, med- | cal, with 1-2 | wings, medium |
| | ium dense, berry | wings, dense, | dense, berry skin |
| | skin with green | berry skin with | with green-yellow |
| | color, firm flesh, | green-yellow color, | color, slightly firm |
| | neutral taste, | slightly firm flesh, | flesh, neutral taste, |
| | no flesh color- | aromatic taste, no | no flesh coloration |
| | ation | flesh coloration | |
| primo- | elliptic cross | circular cross sec- | oblate cross sec- |
| cane and | section, | tion, brownish col- | tion, brownish |
| floricane | brownish color, | or, lenticels: absent, | color, lenticels: |
| | lenticels: absent, | short-medium | absent, medium |
| | short-medium | internodes, diam- | internodes, diam- |
| | internodes, di- | eter small (about | eter small (about |
| | ameter: small | 8 mm) | 8 mm) |
| | (about 8 mm) | | |
| harvest - | early (late Au- | medium (early | Early (late August, |
| ing time | gust in Middle | September in Mid- | Middle Friuli, |
| | Friuli, North- | dle Friuli, North- | North-Eastern |
| | Eastern Italy), | Eastern Italy) | Italy) |
| resis- | resistant to | Average resistance | resistant to winter |
| tances | winter temper- | to winter tempera- | temperature not |
| | ature (−22° C.), | ture (−15° C.), sus- | known, resistant |
| | resistant to | ceptible to downy | to downy mildew, |
| | downy mildew, | mildew, susceptible | resistant to pow- |
| | tolerant to pow- | to powdery mildew | dery mildew |
| | dery mildew, | | |
| | when overripe | | |
| | susceptible to | | |
| | cluster rots. | | |

Of the many commercial cultivars known to the present inventor, the most similar to the new grapevine ‘EARLY SAUVIGNON’ is the female parent ‘Sauvignon’, to which a comparison has been provided above.

### BRIEF DESCRIPTION OF THE PHOTOGRAPHS

The accompanying photographs illustrate the overall appearance of the new grapevine ‘EARLY SAUVIGNON’ showing the colors as true as is reasonably possible with colored reproductions of this type. Colors in the photographs may differ slightly from the color values cited in the detailed botanical description, which accurately describe the color of ‘EARLY SAUVIGNON’.

FIG. 1A and FIG. 1B show typical fruit clusters of ‘EARLY SAUVIGNON’, taken on Aug. 30, 2012 in Udine, Italy.

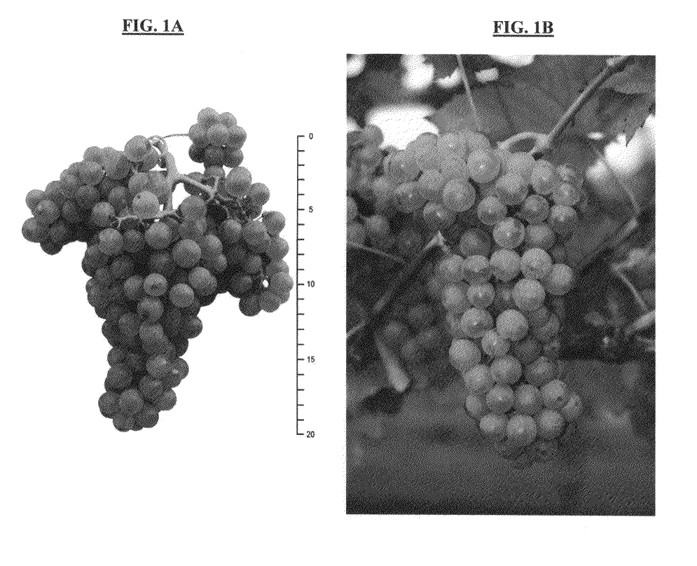

FIG. 2 shows a typical mature leaf (upper surface, left and lower surface, right) of ‘EARLY SAUVIGNON’, taken on Jun. 1, 2012 in Udine, Italy.

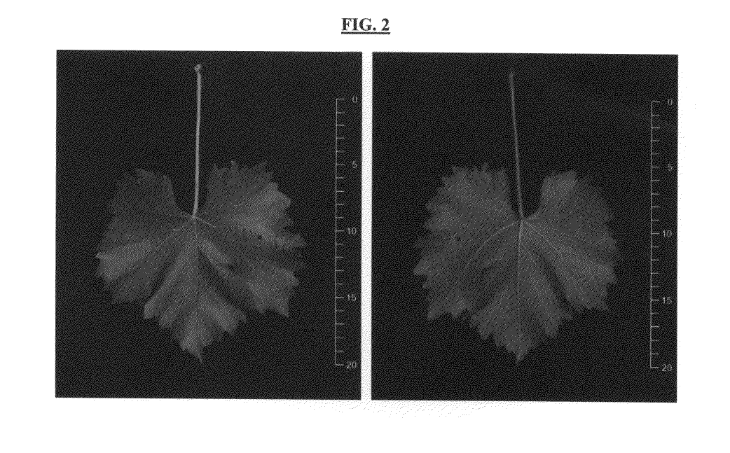

FIG. 3 shows a typical mature vine of ‘EARLY SAUVIGNON’, taken on Aug. 3, 2012.

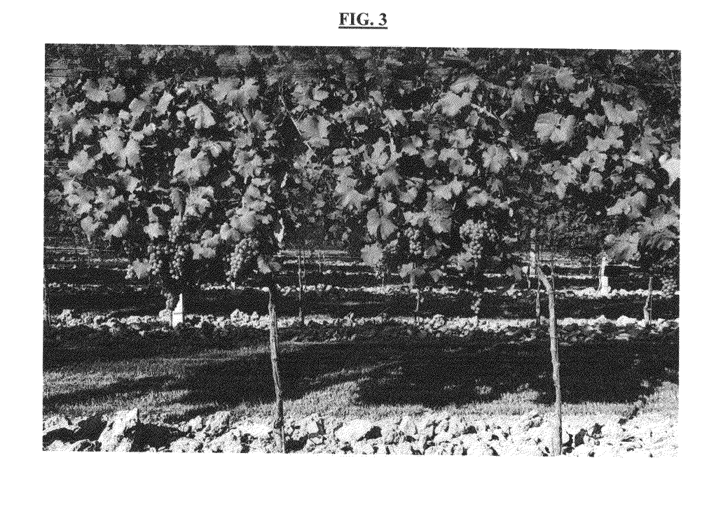

FIG. 4 shows a close-up view of typical mature fruit of ‘EARLY SAUVIGNON’, taken on Aug. 30, 2012 in Udine, Italy

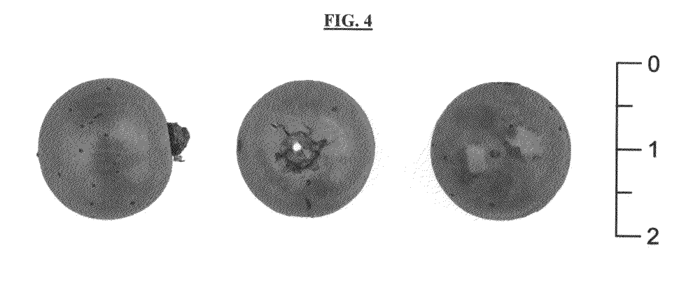

### DETAILED BOTANICAL DESCRIPTION

The following description of ‘EARLY SAUVIGNON’ unless otherwise noted, is based on observations taken during the 2011, 2012, and 2013 growing seasons in Udine, Italy and Fossalon di Grado (GO), Italy, from plants dug from a nursery located in Vivai Cooperativi Rauscedo, Rauscedo (PN), Italy during the beginning of December 2007 and planted approximately 16 to 20 weeks later in Udine, Italy and Fossalon di Grado (GO), Italy. The phenotypical descriptions and color designations stated for the new variety may vary, depending upon variations in environmental factors, including weather (temperature, humidity and light intensity), day length, soil type, location and cultural conditions. ‘EARLY SAUVIGNON’ has not been observed under all possible environmental conditions.

TABLE 3
Detailed Botanical Description

| | | |
|---|---|---|
| | ‘EARLY | Sauvignon |
| Characteristic | SAUVIGNON’ | (unpatented) |

GENERAL

| | | |
|---|---|---|
| Resistance to | Resistant to downy | Susceptible to downy |
| pest/disease | mildew, tolerant to | mildew and to pow- |
| | powdery mildew | dery mildew |
| Resistance to | Resistant to winter | Resistant to winter |
| natural elements | cold down to −22° C. | cold down to −15° C. |

VINE

| | | |
|---|---|---|
| vigor | strong | medium |
| Trunk diameter | 6.5 | 6.5 |
| Bark | | |
| color | Dark brown | Dark brown |
| underbark color | Light brown | Light brown |
| texture | n.a. | n.a. |
| Canes | | |
| length | Internode length | Internode length |
| | cm 9 | about cm 11 |
| Width (diameter) | 8 mm | 8 mm |
| Shoots | | |
| shape | Erected, cross section: | Semi-erected, cross |
| | oblate, surface: with | section: circular, surface: |
| | stripes, nodes and | smooth, nodes and inter- |
| | internodes without | nodes without hairs |
| | hairs | |
| color | internodes with red | Internodes with green |
| | and green pigmentation | color on ventral and |
| | on both sides, nodes | dorsal side, nodes with |
| | with red and green dor- | green ventral and dorsal |
| | sal side and green ven- | side |
| | tral side | |
| Tendrils | | |
| form | Bifid or trifid | bifid |
| color | Green | Green |
| texture | normal | normal |
| number | 2 or <2 consecutive | 2 or <2 consecutive |
| Buds | | |
| size | average | average |
| shape | round | round |
| color | Light brown | Light brown |
| number | 2/node | 2/node |
| time of budbreak | early | early-medium |

LEAVES

| | | |
|---|---|---|
| Size | small-medium | medium |
| Number of leaflets | 5 | 5 |
| Glossiness | Medium-high | medium |
| Cross section | V-shaped | V-shaped |
| shape | | |
| Color (immature) | | |
| Upper surface | Pale green | Pale green |
| Under surface | Pale green | Pale green |
| Color (mature) | | |
| Upper surface | Medium green | Medium green |
| Under surface | Darker green | Darker green |
| Petiole | | |
| Length (cm) | 6.2 | 6.0 |
| Color (upper | green | green |
| surface) | | |
| Color (under | Sometimes reddish | green |
| surface) | | |
| Stipule orientation | n.a. | n.a. |

FLOWERS

| | | |
|---|---|---|
| Flowering period | End of May | End of May |
| (time of beginning | | |
| of flowering) | | |
| Sex | hermaphrodite | hermaphrodite |
| Size | Average | average |
| Diameter (cm) | 0.3 (stamens) | 0.3 (stamens) |
| Fragrance | average | average |
| Flower number (at | n.a. | n.a. |
| 3^{rd} node from tip | | |
| of lateral mean | | |
| and range) | | |
| Petals | | |
| Length (cm) | n.a. | n.a. |
| Width (cm) | n.a. | n.a. |
| Overall shape | calyptra | calyptra |
| Color (immature) | green | green |
| Upper surface | | |
| Under surface | | |
| Color (mature) | green | green |
| Upper surface | | |
| Under surface | | |
| Sepals | None | None |
| Length (cm) | n.a. | n.a. |
| Width (cm) | n.a. | n.a. |
| Overall shape | n.a. | n.a. |
| Color (immature) | n.a. | n.a. |
| Upper surface | | |
| Under surface | | |
| Color (mature) | n.a. | n.a. |
| Upper surface | | |
| Under surface | | |
| Pedicel | | |
| Length (mm) | 7 | 7 |
| Color | green | green |

FRUIT

| | | |
|---|---|---|
| Primocane time of | August 25th | September 7th |
| fruiting (1^{st} pick) | | |
| Clusters | | |
| cluster weight | Low (g 170) | Low (g 230) |
| cluster shape | conical | conical |
| cluster length | mm 180 | medium |
| avg. berries per | 200 | 220 |
| cluster | | |
| avg. clusters per | 2 | 2 |
| shoot | | |
| Berries | | |
| Berry size | g 1.8 | g 1.0 |
| Berry length (cm) | 1.76 | 1.50 |
| Berry width (cm) | 1.56 | 1.50 |
| Overall shape of | broad ellipsoid | globose |
| berry | | |
| Berry Texture | soft | soft |
| Berry Color | green | green |
| (immature) | | |
| Berry Color | green | green |
| (mature) | | |
| Soluble solids (%) | 22.6 | 21.1 |
| Titratable acidity | 4.6 | 5.7 |
| (as g/L tartaric | | |
| acid) | | |
| Sugar/acid ratio | 4.9 | 3.7 |
| Firmness | soft | soft |
| Seeds | 2-3 | 2-3 |
| Skin cracking? | no | rarely |
| Juice color | transparent | transparent |
| Berry taste | neutral | neutral |
| Eating quality | n.a. | n.a. |
| Berry uses | wine | wine |
| Shipping quality | n.a. | n.a. |

What is claimed is:

1. A new and distinct grapevine, referred to as ‘EARLY SAUVIGNON’, as herein described and illustrated by the characteristics set forth above.
